(19) United States
(12) Patent Application Publication
(10) Pub. No.: US 2005/0272717 A1
(43) Pub. Date: Dec. 8, 2005

(54) Treatment of aromatase inhibitor therapy-related osteoporosis

(75) Inventors: Simon N. Jenkins, Audubon, PA (US); Barry S. Komm, Radnor, PA (US); Pol Boudes, Washington Township, NJ (US); Philip Frost, Morris Township, NJ (US); Matthew L. Sherman, Newton, MA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/033,367

(22) Filed: Jan. 11, 2005

Related U.S. Application Data
(60) Provisional application No. 60/536,035, filed on Jan. 13, 2004.

(51) Int. Cl. A61K 31/55

(52) U.S. Cl. 514/217.08

(57) ABSTRACT

This invention relates to the use bazedoxifene (1-[4-(2-azepan-1-yl-ethoxy)-benzyl]-2-(4-hydroxy-phenyl)-3-methyl-1H-indol-5-ol) in the treatment or inhibition of osteoporosis and osteopenia related to aromatase inhibitor therapy.

### CROSS REFERENCE TO RELATED APPLICATIONS

This application claims priority benefit of U.S. Provisional Application Ser. No. 60/536,035 filed Jan. 13, 2004, which is incorporated by reference herein in its entirety.

### BACKGROUND OF THE INVENTION

This invention relates to the use of bazedoxifene (1-[4-(2-azepan-1-yl-ethoxy)-benzyl]-2-(4-hydroxy-phenyl)-3-methyl-1H-indol-5-ol) in the treatment or inhibition of osteoporosis and osteopenia related to aromatase inhibitor therapy.

Aromatase inhibitors, such as anastrozole and letrozole, are frequently used for the adjuvant treatment of breast cancer and for the long term prevention of breast cancer following tamoxifen treatment. By their mechanism of action these drugs induce or amplify the estrogen deficiency that develops in post-menopausal women. The estrogen deficiency induced by aromatase inhibitors is necessary to combat estrogen-dependent breast cancer, but induces side effects in other tissues, notably the bone. Aromatase inhibitors increase bone resorption, and ultimately induce osteopenia and osteoporosis. This has been demonstrated in clinical studies with an increase in biochemical markers of bone resorption, decrease in bone mineral density as measured by dual x-ray absorptiometry (DXA), and an increase incidence of fractures associated with aromatase inhibitors. Because aromatase inhibitors are prescribed on a long-term basis over many years, treatment to prevent osteopenia and osteoporosis is necessary.

Bazedoxifene is a selective estrogen receptor modulator (SERM) that shows tissue-selective estrogen receptor agonist activity on the skeleton and lipid metabolism, while not stimulating uterine and breast tissues. In preclinical studies, bazedoxifene demonstrates improved compressive bone strength and a histomorphometric profile superior to that of raloxifene. This is supported by phase 11 clinical results, which reveal that bazedoxifene reduces biochemical markers of bone resorption.

These data show that bazedoxifene would potentially demonstrate: a) bone protection at a lower dose than currently marketed SERMs, b) no uterine agonist effect, c) a low potential to induce vasomotor flushes and d) improvement in the serum lipid profile. In addition, data also demonstrate that bazedoxifene inhibits estradiol-stimulated proliferation of breast cancer cells, which suggests that bazedoxifene may also act like other SERMs that are able to reduce the risk of developing breast cancer.

### DESCRIPTION OF THE INVENTION

This invention provides the use of bazedoxifene in a mammal receiving aromatase inhibitor therapy in the treatment or inhibition of osteoporosis and osteopenia, particularly related to the aromatase inhibitor therapy. This invention also provides a combination of bazedoxifene and an aromatase inhibitor useful in the treatment of breast cancer, the inhibition of breast cancer in high risk women, and in inhibiting a recurrence of breast cancer, following an initial remission or cure. This invention also provides a kit comprising a bazedoxifene and an aromatase inhibitor as a combined preparation useful in the treatment or inhibition of the previously described conditions. This invention also provides a composition comprising a bazedoxifene and an aromatase inhibitor wherein the composition is provided in an effective amount to a mammal in need thereof. This invention further provides the administration of bazedoxifene following completion of aromatase inhibitor treatment to ameliorate symptoms or side effects of the aromatase inhibitor treatment.

In one aspect, the present invention provides methods for treating or inhibiting osteoporosis or osteopenia in a mammal receiving aromatase therapy, which comprises providing the mammal an effective amount of bazedoxifene. In some such embodiments, the bazedoxifene is bazedoxifene acetate. In some such embodiments, the bazedoxifene is provided in combination with an aromatase inhibitor. In some such embodiments, the bazedoxifene or the aromatase inhibitor or both are provided in subtherapeutically effective amounts. In some embodiments, the aromatase inhibitor is selected from the group consisting of exemestane, formestane, atamestane, fadrozole, letrozole, vorozole, and anastrozole. In some embodiments, the aromatase inhibitor is selected from the group consisting of letrozole and anastrozole.

In another aspect, the present invention provides methods for treating or inhibiting breast cancer in a mammal receiving aromatase therapy, which comprises providing the mammal an effective amount of bazedoxifene. In some such embodiments, the bazedoxifene is bazedoxifene acetate. In some such embodiments, the bazedoxifene is provided in combination with an aromatase inhibitor. In some such embodiments, the bazedoxifene or the aromatase inhibitor or both are provided in subtherapeutically effective amounts. In some embodiments, the aromatase inhibitor is selected from the group consisting of exemestane, formestane, atamestane, fadrozole, letrozole, vorozole, and anastrozole. In some embodiments, the aromatase inhibitor is selected from the group consisting of letrozole and anastrozole.

In a further aspect, this invention provides methods for treating or inhibiting recurrence of breast cancer in a mammal in need thereof, which comprises providing the mammal an effective amount of bazedoxifene. In some such embodiments, the bazedoxifene is bazedoxifene acetate. In some such embodiments, the bazedoxifene is provided in combination with an aromatase inhibitor. In some such embodiment, the bazedoxifene or the aromatase inhibitor or both are provided in subtherapeutically effective amounts. In some embodiments, the aromatase inhibitor is selected from the group consisting of exemestane, formestane, atamestane, fadrozole, letrozole, vorozole, and anastrozole. In some embodiments, the aromatase inhibitor is selected from the group consisting of letrozole and anastrozole.

Another aspect of the invention provides methods for treating or inhibiting breast cancer in a high risk woman, which comprises providing the woman an effective amount of bazedoxifene. In some such embodiments, the bazedoxifene is bazedoxifene acetate. In some such embodiments, the bazedoxifene is provided in combination with an aromatase inhibitor. In some such embodiments, the bazedoxifene or the aromatase inhibitor or both are provided in subtherapeutically effective amounts. In some embodiments, the aromatase inhibitor is selected from the group consisting of exemestane, formestane, atamestane, fadrozole, letrozole, vorozole, and anastrozole. In some embodiments, the aromatase inhibitor is selected from the group consisting of letrozole and anastrozole.

A further aspect of this invention is to provide methods for administering bazedoxifene to a mammal who has completed aromatase inhibitor treatment to ameliorate symptoms or side effects of the aromatase inhibitor treatment. In some such embodiments, the symptom or side effects are osteopenia or osteoporosis. In some such embodiments, the bazedoxifene is bazedoxifene acetate. In some such embodiments, the bazedoxifene is provided in subtherapeutically effective amounts.

As used in accordance with this invention, the term “bazedoxifene” means bazedoxifene (1-[4-(2-azepan-1-yl-ethoxy)-benzyl]-2-(4-hydroxy-phenyl)-3-methyl-1H-indol-5-ol), or a pharmaceutically acceptable salt or prodrug thereof.

As used in accordance with this invention, the terms “treatment” or “treating” mean curing, ameliorating or reversing the progress of a disease or disorder, or ameliorating or reversing one or more symptoms or side effects of such disease or disorder.

As used in accordance with this invention, the terms “providing” or “provided” with respect to providing the bazedoxifene-aromatase inhibitor combination, mean either directly administering the combination, or administering a prodrug, derivative, or analog of one or both of the components of the combination which will form an effective amount of the combination within the body, and with respect to bazedoxifene, means either directly administering bazedoxifene, or administering a prodrug, derivative, or analog of bazedoxifene which will form an effective amount in the body.

As used in accordance with this invention, the terms “high risk women” or “a high risk woman” mean a woman at least 35 years of age with a 5-year predicted risk of breast cancer >1.67%, as calculated by the Gail Model [The Lancet, 355:18 (2000)].

As used in accordance with this invention, the term “subtherapeutically” means below the dosage levels used normally to treat a disease, i.e., a dosage less than the amount required for a therapeutic effect. When used for the combination therapy of bazedoxifene and an aromatase inhibitor, it may refer to a synergistic effect between the compounds.

Aromatase is an enzyme which converts androgens to estrone. Estrone can subsequently be converted to estradiol, which has been linked to increased growth or proliferation of estrogen receptor positive carcinoma. As used in accordance with this invention, the term “aromatase inhibitor” means compounds or substances which inhibit the activity of the enzyme aromatase. Thus, the goal of using aromatase inhibitors in chemotherapy is typically to reduce the levels of circulating estradiol, to ultimately inhibit the growth of neoplasms that are estrogen receptor positive or estrogen dependent. There are two types of aromatase inhibitors: steroidal (type 1) and non-steroidal (type 11) inhibitors. Examples of steroidal aromatase inhibitors include exemestane, formestane, atamestane, and the like. Examples of non-steroidal aromatase inhibitors include fadrozole, letrozole, vorozole, anastrozole, and the like.

The preparation of bazedoxifene and its pharmaceutically acceptable salts is described in U.S. Pat. No. 5,998,402, which is hereby incorporated by reference in its entirety. When administered for the treatment or inhibition of a particular disease state or disorder, it is understood that the effective dosage may vary depending upon, e.g., whether bazedoxifene is utilized with or without an aromatase inhibitor, the particular aromatase inhibitor utilized, if one is used, the mode of administration, the condition being treated, and severity thereof, as well as the various physical factors related to the individual being treated. Effective administration of the bazedoxifene of this invention may be in any of a variety of dosage regimes such as single dosage, multiple dosage, combination dosage and delay or time release dosage forms. Bazedoxifene acetate has been evaluated in clinical trials using dosages of 10, 20, and 40 mg/day. Based on the results of these trials, it is anticipated that doses between about 5 and about 80 mg/day of bazedoxifene will provide an effective amount within the methods of the invention. The projected daily dosages are expected to vary with route of administration. The selection of the appropriate administration and dosage forms for an individual patient will be apparent to those skilled in the art. Such determinations are routine to one of ordinary skill in the art (see for example, Harrison's Principles of Internal Medicine (1998), edited by Anthony Fauci et al., 14^{th} edition, published by McGraw Hill).

In some embodiments, a combination of bazedoxifene and an aromatase inhibitor is administered in accordance with the invention wherein the aromatase inhibitor is letrozole, and wherein both are provided orally. In some such embodiments, the initial oral dosage of bazedoxifene can be from about 5 to about 80 mg/day (on days that it is provided) and the initial oral dose of letrozole can be from about 1 to about 10 mg daily (on days that it is provided). In some such embodiments, the initial oral dosage of bazedoxifene can be from about 10 to about 60 mg/day (on days that it is provided) and the initial oral dose of letrozole can be from about 1 to about 10 mg daily (on days that it is provided). In some such embodiments, the initial oral dosage of bazedoxifene can be from about 10 to about 40 mg/day (on days that it is provided) and the initial oral dose of letrozole can be from about 1 to about 10 mg daily (on days that it is provided). Further, in some such embodiments, the initial oral dosage of bazedoxifene can be from about 10 to about 30 mg/day (on days that it is provided) and the initial oral dose of letrozole can be from about 1 to about 10 mg daily (on days that it is provided). In some such embodiments, the initial oral dosage of bazedoxifene can be from about 20 to about 40 mg/day (on days that it is provided) and the initial oral dose of letrozole can be from about 1 to about 10 mg daily (on days that it is provided).

In some embodiments, a combination of bazedoxifene and an aromatase inhibitor is administered in accordance with the invention wherein the aromatase inhibitor is anastrozole, and wherein both are provided orally. In some such embodiments, the initial oral dosage of bazedoxifene can be from about 5 to about 80 mg/day (on days that it is provided) and the initial oral dose of anastrozole can be from about 1 to about 10 mg daily (on days that it is provided). In some such embodiments, the initial oral dosage of bazedoxifene can be from about 10 to about 60 mg/day (on days that it is provided) and the initial oral dose of anastrozole can be from about 1 to about 10 mg daily (on days that it is provided). In some such embodiments, the initial oral dosage of bazedoxifene can be from about 10 to about 40 mg/day (on days that it is provided) and the initial oral dose of anastrozole can be from about 1 to about 10 mg daily (on days that it is provided). In some such embodiments, the initial oral dosage of bazedoxifene can be from about 10 to about 30 mg/day (on days that it is provided) and the initial oral dose of anastrozole can be from about 1 to about 10 mg daily (on days that it is provided). In some such embodiments, the initial oral dosage of bazedoxifene can be from about 20 to about 40 mg/day (on days that it is provided) and the initial oral dose of anastrozole can be from about 1 to about 10 mg daily (on days that it is provided).

The aromatase inhibitors specifically named in this disclosure are commercially available or can be made from known procedures in the literature. This invention is not limited to the use of the specific aromatase inhibitors named herein; the preparation of other aromatase inhibitors will be apparent to one skilled in the art based on the literature.

As used in this invention, the combination therapy can be given simultaneously or can be given in a staggered regimen, with bazedoxifene being given at a different time than the aromatase inhibitor. This time differential may range from several minutes, hours, days, weeks, or longer between administration of the two agents. Therefore, the term “combination” or “combined” does not necessarily mean administered at the same time or as a unitary dose, but that each of the components are administered during a desired treatment period. The agents may be administered by the same or different routes. For example, one component may be administered orally, while the other parenterally and these combinations can be administered daily, weekly, or even once monthly.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface-modifying agents include nonionic and anionic surface-modifying agents. Representative examples of surface-modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is nontoxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

For bazedoxifene, solid oral formulations, such as in the form of a film coated tablet or capsule, useful for this invention include the active pharmacological agents disclosed herein in combination with carrier or excipient systems having the components:
a) a filler and disintegrant component comprising from about 5% to about 82% by weight (wght) of the total formulation, preferably between about 30% and about 80% of the formulation, of which from about 4% to about 40% by weight of the total formulation comprises one or more pharmaceutically acceptable disintegrants;
b) optionally, a wetting agent comprising from about 0.2 to about 5% of the composition (wght), such as selected from the group of sodium lauryl sulfate, polyoxyethylene sorbitan fatty acid esters, polyoxyethylene alkyl ethers, sorbitan fatty acid esters, polyethylene glycols, polyoxyethylene castor oil derivatives, docusate sodium, quaternary ammonium compounds, sugar esters of fatty acids and glycerides of fatty acids;
c) a lubricant comprising from about 0.2% to about 10% of the composition (wght), such as selected from the group of magnesium stearate or other metallic stearates (e.g. calcium stearate or zinc stearate), fatty acid esters (e.g. sodium stearyl fumarate), fatty acids (e.g. stearic acid), fatty alcohols, glyceryl behenate, mineral oil, paraffins, hydrogenated vegetable oils, leucine, polyethylene glycols, metallic lauryl sulfates and sodium chloride; and
d) optionally, a glidant comprising from about 0.1% to about 10% (wght) of the composition, the glidant selected from those known in the art, including from the group of silicon dioxide, talc, metallic stearates, calcium silicate or metallic lauryl sulfates.

While the formulations described herein may be used in an uncoated or non-encapsulated solid form, in some embodiments the final compositions are coated or encapsulated. The pharmacological compositions may be optionally coated with a film coating, e.g., comprising from about 0.3% to about 8% by weight of the overall composition. Film coatings useful with the present formulations are known in the art and generally consist of a polymer (usually a cellulosic type of polymer), a colorant and a plasticizer. Additional ingredients such as wetting agents, sugars, flavors, oils and lubricants may be included in film coating formulations to impart certain characteristics to the film coat. The compositions and formulations herein may also be combined and processed as a solid, then placed in a capsule form, such as a gelatin capsule.

The filler component listed above may utilize the filler or binder components known in the art for solid oral formulations. Pharmaceutically acceptable fillers or binding agents can be selected from those known in the art such as, but not limited to, lactose, microcrystalline cellulose, sucrose, mannitol, calcium phosphate, calcium carbonate, powdered cellulose, maltodextrin, sorbitol, starch, or xylitol.

In conjunction with or in place of the materials listed above for the filler component, the present formulations utilize disintegrant agents. These disintegrants may be selected from those known in the art, including pregelatinized starch and sodium starch glycolate. Other useful disintegrants include croscarmellose sodium, crospovidone, starch, alginic acid, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein will comprise from about 4% to about 40% of the composition by weight, preferably from about 15% to about 35%, more preferably from about 20% to about 35%. Some components may have multiple functions in the formulations of this invention, acting e.g. as both a filler and a disintegrant. Such a component may be referred to as a filler disintegrant and its function in a specific formulation may be singular even though its properties may allow multiple functionality.

The pharmaceutical formulations and carrier or excipient systems herein may also contain an antioxidant or a mixture of antioxidants, e.g., ascorbic acid. Other antioxidants which may be used include sodium ascorbate and ascorbyl palmitate, which may be used in conjunction with an amount of ascorbic acid. In some embodiments, the amount of antioxidant(s) is from about 0.5% to about 15% by weight. In some embodiments, the amount of antioxidants is from about 0.5% to about 5% by weight.

Among the formulations of this invention are pharmaceutical formulations containing a pharmaceutically effective amount of an active pharmacological agent and a carrier or excipient system comprising:
a) a filler and disintegrant component comprising between about 50% and about 87% of the formulation, with from about 4% to about 40% of the formulation comprising one or more disintegrant agents;
b) a wetting agent comprising between about 0.5% and about 2.7% of the formulation;
c) a lubricant comprising between about 0.2% and about 5.5% of the formulation; and
d) a glidant comprising between about 0.1% and about 5.5% of the formulation.

The percentages listed in the formulations above indicate percentages by weight of the total weight of the components listed from a) to d). The formulations above may also contain an optional antioxidant component, e.g., ascorbic acid, at a concentration of from about 0.5% to about 5.5% by weight of the formulation. The formulations may be contained within a pharmaceutically acceptable capsule, such as a gel capsule, or coated with a film coating comprising from about 0.3% to about 8% by weight of the formulation.

This invention also comprises a pharmaceutical carrier or excipient systems useful in pharmaceutical compositions utilizing as an active ingredient one or more of the compounds described herein, or a pharmaceutically acceptable salt thereof, as described herein. These pharmaceutical carrier or excipient systems comprise, by weight:
a) a filler and disintegrant component comprising between about 54% and about 80% of the formulation, with the disintegrant agent(s) therein comprising from about 4% to about 40% by weight of the overall formulation;
b) a wetting agent comprising between about 0.55% and about 2.5% of the formulation;
c) a lubricant comprising between about 0.2% and about 5.5% of the formulation; and
d) a glidant comprising between about 0.1% and about 5.0% of the formulation.

In some embodiments the carrier or excipient systems above also optionally contain an antioxidant component, e.g., ascorbic acid, at a concentration of from about 0.1% to about 5.0% by weight.

Among the carrier or excipient systems of this invention are those comprising:
a) a filler and disintegrant component, as described above, comprising between about 50% and about 87% of the formulation, the disintegrant(s) therein comprising from about 25% to about 35% of the formulation, by weight;
b) a wetting agent comprising between about 0.55% and about 2.7% of the formulation;
c) a lubricant comprising between about 0.2% and about 5.5% of the formulation;
d) a glidant comprising between about 0.1% and about 5.5% of the formulation; and
e) an antioxidant component, e.g., ascorbic acid, at a concentration of from about 0.1% to about 5.5% by weight.

### EXAMPLES

### Example 1

BAZEDOXIFENE ACETATE - RAPID
DISSOLUTION FORMULATIONS

| | without | with |
|---|---|---|
| | Ascorbic | Ascorbic |
| Ingredient | Acid | Acid |
| Bazedoxifene acetate, | 10.00 | 10.00 |
| micronized* | | |
| Lactose NF fast flow | 33.10 | 31.60 |
| Microcrystalline Cellulose, NF | 25.00 | 25.00 |
| (Avicel PH101) | | |
| Starch 1500 | 20.00 | 20.00 |
| Sodium Lauryl Sulfate NF | 1.50 | 1.50 |
| Sodium Starch Glycolate | 10.00 | 10.00 |
| Ascorbic Acid USP | — | 1.5 |
| Syloid 244 FP | 0.15 | 0.15 |
| Magnesium Stearate | 0.25 | 0.25 |

*Amount in formula is adjusted for actual potency of bazedoxifene as free base. Corresponding adjustment made with lactose.

The formulations given above in Table 1 were prepared by incorporating a portion of the excipients in the granulation and a portion is also added in the final blending steps as dry powders. A dissolution profile generated for the formulations demonstrated almost 90% release of the drug in 30 minutes. Thus, the unique combination of disintegrants and soluble diluents plus the incorporation of both granulated and powdered solids into the composition ensures the fastest release of drug.

Wet granulation of the formulations as described in Table 1 may be carried out by mixing the drug and ascorbic acid with a portion of the lactose, microcrystalline cellulose, pregelatinized starch and sodium starch glycolate. The sodium lauryl sulfate is dissolved in the water and used to granulate the mixture of powders in a high shear mixer. The granulation is dried in a fluid bed dryer to a moisture of 2-3%. The particle size of the dried granulation is controlled by passing through a mill equipped with knife-edged blades and using a 20- or 30-mesh screen. The silicon dioxide and remaining lactose, microcrystalline cellulose, pregelatinized starch, and sodium starch glycolate are mixed with the milled granulation in a tumble-type mixer. The final blend is prepared by adding magnesium stearate to the tumble-type mixer and mixing. Compression is carried out on a rotary tablet press using appropriate size tooling. Coating is performed in conventional coating pans and by applying the coating suspension to achieve a suitable film coat.

### Example 2

MODIFIED BAZEDOXIFENE ACETATE FORMULATION
% w/w

| | 5% |
|---|---|
| Ingredient | granulation |
| Bazedoxifene acetate, micronized^{a} | 5.00 |
| Lactose NF | 41.00 |
| Microcrystalline Cellulose, NF | 35.00 |
| Pregelatinized Starch NF | 10.00 |
| Sodium Lauryl Sulfate NF | 1.50 |
| I-Ascorbic Acid USP | 1.50 |
| Sodium Starch Glycolate NF | 5.50 |
| Magnesium Stearate NF | 0.50 |
| Pur. Water USP^{b} | qs |

^{a}Amount in formula is adjusted for actual potency of bazedoxifene acetate as free base. Corresponding adjustment made with lactose.
^{b}Used in process but does not appear in the final product.

### Example 3

### Bazedoxifene Acetate at 5% Granulation

A preferred carrier or excipient system for formulating a granulation of from about 2 to about 8% by weight of one of the active pharmacological agents of this invention, e.g., about 5%, may be produced utilizing the carrier or excipient components on a weight percentage; lactose from about 32% to about 38%, microcrystalline cellulose from about 32% to about 38%, pregelatinized starch from about 12% to about 16%, ascorbic acid from about 1% to about 2%, sodium lauryl sulfate from about 1% to about 2%, sodium starch glycolate from about 4% to about 8%, silicon dioxide from about 0.1% to about 0.2% and magnesium stearate from about 0.3% to about 0.7%.

A formulation of this invention utilizing bazedoxifene as the active ingredient at a 5% granulation was prepared utilizing the components listed below in a granulation part and a dry part of components.

| | | |
|---|---|---|
| Item No. | Ingredients | Mg/Unit |

Granulation Part:

| | | |
|---|---|---|
| 1 | Bazedoxifene acetate | 5.00 |
| 2 | Lactose NF | 26.60 |
| 3 | Microcrystalline Cellulose NF | 25.00 |
| 4 | Pregelatinized Starch NF | 10.00 |
| 5 | Ascorbic Acid USP | 1.50 |
| 6 | Sodium Lauryl Sulfate NF | 1.50 |
| 7 | Sodium Starch Glycolate NF | 4.00 |
| 8 | Water, Purified USP | Q.S. |
| | | 73.60 |
| Dry Part: | | |
| 9 | Lactose NF (fast flo) | 9.75 |
| 10 | Microcrystalline Cellulose NF | 10.00 |
| 11 | Pregelatinized Starch NF | 4.00 |
| 12 | Sodium Starch Glycolate NF | 2.00 |
| 13 | Silicon Dioxide NF | 0.15 |
| 14 | Magnesium Stearate NF | 0.50 |
| | | 100.00 |

A film coat of White Opadry I (YS-1-18027-A) was applied to the tablets, which were compressed as follows:

| Dose of Bazedoxifene | tablet weight, mg | mg of film coat applied/tablet |
|---|---|---|
| 5 mg | 100 | 6.0 |
| 10 mg | 200 | 8.0 |
| 20 mg | 400 | 13.0 |

### Example 4

### Bazedoxifene in the Treatment of Osteopenia or Osteoporosis

Mice, rats or monkeys may be treated daily with an aromatase inhibitor and bazedoxifene combination. Administration of both components of the combination may be by subcutaneous, intraperitoneal or oral routes for periods ranging from 2 weeks to one year. Bone mineral density may be measured by standard methods, e.g., dual-energy x-ray absorptiometry (Wickman, S. et al. J. Clin. Endocrin. & Metab. 88(8):3785-3793 (2003); Vanderschueren, D. et al. Endocrin. 138(8):2310-2307 (1997)). Uterine weights may be measured as described by Schieweck, K. et al. in J. Steroid Biochem. Mol. Biol. 44(4-6):633-6 (1993). A decrease in uterine weight is a positive control, ensuring aromatase functional activity. An improvement in bone mass loss following co-treatment with bazedoxifene would demonstrate that the bazedoxifene is effective in treating bone mass loss by maintaining bone mass without stimulating the uterus or breast tissues.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention. Each reference cited in the present application, including literature references, books, patents and patent applications, is incorporated herein by reference in its entirety.

1. A method of treating or inhibiting osteoporosis or osteopenia in a mammal receiving aromatase therapy, which comprises providing to said mammal an effective amount of bazedoxifene.

2. A method according to claim 1 wherein the bazedoxifene is provided in combination with an aromatase inhibitor.

3. A method according to claim 1, wherein the aromatase inhibitor is selected from the group consisting of exemestane, formestane, atamestane, fadrozole, letrozole, vorozole, and anastrozole.

4. A method according to claim 3, wherein the aromatase inhibitor is letrozole or anastrozole.

5. A method according to claim 1, wherein the bazedoxifene or the aromatase inhibitor or both are provided in subtherapeutically effective amounts.

6. A method according to claim 1, wherein the bazedoxifene is bazedoxifene acetate.

7. A method of treating or inhibiting breast cancer in a mammal in need thereof, which comprises providing to said mammal an effective amount of bazedoxifene and an aromatase inhibitor.

8. A method according to claim 7, wherein the aromatase inhibitor is selected from the group consisting of exemestane, formestane, atamestane, fadrozole, letrozole, vorozole, and anastrozole.

9. A method according to claim 8, wherein the aromatase inhibitor is letrozole or anastrozole.

10. A method according to claim 7, wherein the bazedoxifene or the aromatase inhibitor or both are provided in subtherapeutically effective amounts.

11. A method according to claim 7, wherein the bazedoxifene is bazedoxifene acetate.

12. A method of inhibiting the recurrence of breast cancer in a mammal in need thereof which comprises providing to said mammal an effective amount of bazedoxifene and an aromatase inhibitor.

13. A method according to claim 12, wherein the aromatase inhibitor is selected from the group consisting of exemestane, formestane, atamestane, fadrozole, letrozole, vorozole, and anastrozole.

14. A method according to claim 13, wherein the aromatase inhibitor is letrozole or anastrozole.

15. A method according to claim 12, wherein the bazedoxifene or the aromatase inhibitor or both are provided in subtherapeutically effective amounts.

16. A method according to claim 12, wherein the bazedoxifene is bazedoxifene acetate.

17. A method of inhibiting breast cancer in a high risk woman, which comprises providing to said woman, an effective amount of bazedoxifene and an aromatase inhibitor.

18. A method according to claim 17, wherein the aromatase inhibitor is selected from the group consisting of exemestane, formestane, atamestane, fadrozole, letrozole, vorozole, and anastrozole.

19. A method according to claim 18, wherein the aromatase inhibitor is letrozole or anastrozole.

20. A method according to claim 17, wherein the bazedoxifene or the aromatase inhibitor or both are provided in subtherapeutically effective amounts.

21. A method according to claim 17, wherein the bazedoxifene is bazedoxifene acetate.

22. A method according to claim 1, wherein the effective amount of bazedoxifene is from about 5 to about 80 mg/day.

23. A method according to claim 22, wherein the effective amount of bazedoxifene is from about 10 to about 60 mg/day.

24. A method according to claim 23, wherein the effective amount of bazedoxifene is from about 10 to about 40 mg/day.

25. A method according to claim 24, wherein the effective amount of bazedoxifene is from about 10 to about 30 mg/day.

26. A method according to claim 24, wherein the effective amount of bazedoxifene is from about 20 to about 40 mg/day.

27. A composition comprising a bazedoxifene and an aromatase inhibitor, wherein said composition is provided in an effective amount to a mammal in need thereof.

28. A composition according to claim 27, wherein the bazedoxifene is in a effective amount from about 5 to about 80 mg/day.

29. A composition according to claim 28, wherein the bazedoxifene is in a effective amount from about 10 to about 60 mg/day

30. A composition according to claim 29, wherein the bazedoxifene is in a effective amount from about 10 to about 40 mg/day.

31. A composition according to claim 30, wherein the bazedoxifene is in a effective amount from about 10 to about 30 mg/day.

32. A composition according to claim 30, wherein the bazedoxifene is in a effective amount from about 20 to about 40 mg/day.

33. A kit comprising a bazedoxifene and an aromatase inhibitor as a combined preparation for simultaneous, separate or sequential use in the treatment or inhibition of osteoporosis, osteopenia or breast cancer in a mammal.

34. A kit comprising a bazedoxifene and an aromatase inhibitor as a combined preparation for simultaneous, separate or sequential use in inhibiting breast cancer in a high risk woman, or in inhibiting the recurrence of breast cancer in a mammal.
